(19) United States
(12) Patent Application Publication
(10) Pub. No.: US 2014/0356410 A1
(43) Pub. Date: Dec. 4, 2014

(54) BIODEGRADABLE AND BIOCOMPATIBLE NANO COMPOSITE T-PLATE IMPLANT AND A METHOD OF SYNTHESIZING THE SAME

(71) Applicants: Jafar Ai, TEHRAN (IR); Nastaran Elyasi far, Tehran (IR); Mahmoud Azami, Tehran (IR); Naghmeh Bahrami, Tehran (IR); Shima Tavakol, Tehran (IR)

(72) Inventors: Jafar Ai, TEHRAN (IR); Nastaran Elyasi far, Tehran (IR); Mahmoud Azami, Tehran (IR); Naghmeh Bahrami, Tehran (IR); Shima Tavakol, Tehran (IR)

(21) Appl. No.: 14/301,306

(22) Filed: Jun. 10, 2014

(51) Int. Cl. A61L 31/12; C12N 5/071; A61K 35/48

(52) U.S. Cl. CPC A61L 31/127; A61K 35/48; C12N 5/0682; C12N 2533/40
USPC 424/426; 424/93.7; 435/180

(57) ABSTRACT

The embodiments herein provide a biodegradable and biocompatible T-plate nano-composite implant with stem cells for treating and repairing broken bones, damaged tissues and torn ligaments. The implant comprises a polymeric matrix part comprising poly lactic glycolic acids (PLGA), a bioceramic part comprising hydroxyapatite (HAp) nanoparticles and an endometrial stem cell. The PLGA and HAp nanoparticles act as a matrix and reinforcing agents respectively. A method is provided for synthesizing the T-plate implant. The method comprises synthesizing hydroxyapatite (HAp) nanoparticles, poly lactic glycolic acids (PLGA) and HAp nano composite implant. The casting of the poly lactic glycolic acids (PLGA) and HAp nano composite are done in a mold to obtain a T-plate nano composite. An endometrial stem cell from an epithelial cell lining from uterus is isolated and cultured. The endometrial stem cells are implanted on the nano-composite implant.

### BACKGROUND

1. Technical Field

The embodiments herein generally relate to a field of medical device. The embodiments herein particularly relate to an implant used in a repair of broken bones and torn ligament. The embodiments herein more particularly relate to a biodegradable and biocompatible nano composite implant used in treatment of fractures, injuries and tissue damage.

2. Description of the Related Art

Broken bones, torn ligaments and damaged tissues are the medical condition in which there is a break in the continuity of tissues. The tissue damage or fractures are a result of high force impact/stress such as accident. The tissue damage or fractures also arise due to medical conditions such as osteoporosis, bone cancer, osteogenesis imperfecta and arthritis.

The diagnosis and treatment of fractured bones, broken bones, torn ligaments and damaged tissues is initiated by X-ray scan or MRI scan. The scanning result and physical examination by the doctor reveals the severity of the tissue damage. The treatment and remedial measures are selected based on the severity and the patients need. Depending on the type and severity of the fractures or tissue damage, a back-slab or a partial cast is applied to stabilize and support the broken bone/limb tissue and to support the surrounding muscles. This is known as the primary immobilization.

The treatment methods mainly include a primary cast and a secondary cast. The primary cast is applied to immobilize the area and allow healing of fractured bone. The primary casts are used mainly in minor fractures or tissue tear or strain. The primary casts are used to stabilize bone fractures. The primary casts keep the fractured or broken tissue and the surrounding muscles in a stable position. More complicated fractures or multiple fractures require surgery. In case of multiple fractures, the patient needs a metal rod or a metal implant placed at the centre of the bone. In case of compound fractures, metal rods or plates are placed outside the limb. The secondary cast is intended to provide a full support for the injured throughout a healing process.

In the several surgical techniques to repair the broken bones and torn ligaments, the metallic implants are commonly used. The metallic implants mainly include metallic plates and screws. The long term effects of the metal plates and screws for the body is harmful and a second surgery is required to remove the metal plates and screws. The notable complications include pain due to tissue abrasion, hypersensitivity due to metals such as titanium, temperature rise due to interaction with radiation and imaging, stress shielding, growth restriction for kids, infection and sudden pressure during removal of the metal implants.

The metallic implants cause a wide variety of problems. The metallic implants are exposed to aqueous environment of the body and the metal implants undergo corrosion. The products of metal corrosion cause immunological problems and threaten the biocompatibility of implants. The released metallic ions due to corrosion have a carcinogenic effect on the body cells. Stress shield is another problem of metallic implants. The stress shield event happens for most of metallic implants due to considerable difference between elastic modulus of metal implant and natural bones.

The principal disadvantage of metal implant is their corrosion tendency in the in-vivo environment. Most metals are tolerated by the human body in small amounts in the form of metallic ions. The corrosion and the disintegration of the metal implant weaken the implant and leads to harmful effect of corrosion products on the surrounding tissues and organs. Also the metal T-plates or implants makes the patients undergo secondary surgery to remove implant after repair of bone crack.

Other disadvantages are bone resorption caused by bone plates and screws, which carry most of the external loads, leading to stress protection produced by the modulus mismatch between metals and bone. Another disadvantage of the metal implants is the carcinogenic potential and the corrosion by product release. Therefore, surgeons are recommended to remove metallic implants in a second operation once the fracture or tissue damage has healed.

Another type of metallic implants are bioinert in nature. The bioinert material in the body cannot bond with bones and the final response of body to these implants is a formation of the fibrous tissue around the implant.

The metal implants cause the pathogenesis due to the reactivity with steroids, alcohol, genetic factors and hyper-coagulability. In 2012, intermediary pins for fixation of fractured zone came into wide use for animals. The technique had a lot of problems. Later on the researchers used the method of internal fixation. Based on the pin fixation method, the damaged medulla and cortex are fixed. The pin fixation method is used to minimize the damage to the soft tissues.

The use of biodegradable composite polymer/bioceramic is a solution for the above mentioned problems. The new biodegradable polymers such as poly glycolic lactic acid (PLGA), and calcium phosphate ceramics, provide a better control of the macroscopic and microscopic composite structures for improving the bone defects. The biodegaradable polymers are also used to reduce the brittleness of conventional ceramics. The biodegradable polymer composites/ceramic biomaterial is promising for bone grafting. Also the application of bone marrow stem cells along with biodegradable polymer/ceramic composite accelerates the bone healing process.

Hence there is a need for a non-toxic, biodegradable ceramic/polymer composite implant for a treatment of broken or fractured bones. Also there is a need for a biodegradable implant in combination with stem cells for early recovery of broken or fractured bones. Further there is a need for a biodegradable composite implant in combination with the stem cells to eliminate a need of multiple surgeries for a removal of an implant. Also there is a need for a biodegradable composite implant with no stress shielding effects.

The above mentioned shortcomings, disadvantages and problems are addressed herein and which will be understood by reading and studying the following specification.

### OBJECTIVES OF THE EMBODIMENTS

The primary object of the embodiments herein is to provide a simple and cost effective therapy involving biodegradable polymer/bioceramic composite structure for treating a bone defect or tissue damage.

Another object of the embodiments herein is to provide a combination of stem cells with biodegradable composite/polymer/bioceramic composite structure for accelerated a healing of tissue damage.

Yet another object of the embodiments herein is to provide a biodegradable and biocompatible polymer/bioceramic composite with a Young's Modulus of 157 MPa.

Yet another object of the embodiments herein is to provide a biodegradable and biocompatible implant for eliminating a secondary surgery for the removal of implants after the damaged tissue or bone defect has healed.

Yet another object of the embodiments herein is to provide a biodegradable and biocompatible implant with a controlled rate of degradability and mechanical properties.

Yet another object of the embodiments herein is to provide the biodegradable and biocompatible implants for an accelerated bone damage healing.

Yet another object of the embodiment herein is to provide a biodegradable and biocompatible implant with an osteo-conductive property.

Yet another objective of the embodiment herein is to provide a biodegradable and biocompatible implant composite, which prevents increased acidity in the injured tissue area.

Yet another objective of the embodiment herein is to provide a biodegradable and biocompatible implant composite composed of a polymer and bioceramic material.

Yet another objective of the embodiments herein is to provide a biodegradable and biocompatible implant composite which is environment friendly and non toxic.

Yet another objective of the embodiments herein is to provide a biodegradable and biocompatible implant composite with endometrial stem cells compatible with biodegradable copolymer.

Yet another objective of the embodiments herein is to provide a biodegradable and biocompatible implant composite for maintaining a variety of fractures.

These and other objects and advantages of the embodiments herein will become readily apparent from the following detailed description taken in conjunction with the accompanying drawings.

### SUMMARY

The various embodiments herein provide a biodegradable implant composite for the treatment of injured/damaged tissue especially fractured bones. The biodegradable implant along with stem cells accelerates the healing of the injured/broken tissue such as bone.

A biodegradable and biocompatible nano-composite T-plate implant comprises a polymeric matrix part, a bioceramic part and an endometrial stem cell. The polymeric matrix part comprises poly lactic glycolic acids (PLGA), and the bioceramic part comprises hydroxyapatite (HAp) nanoparticles. The poly lactic glycolic acid (PLGA) acts as a matrix and the hydroxyapatite (HAp) nanoparticles act as reinforcing agents.

According to an embodiment herein, the hydroxyapatite (HAp) nanoparticles have a spherical shape or a needle like shape within the polymeric matrix part.

According to an embodiment herein, the T-plate nano-composite implant has a Young's modulus of 157 MPa.

According to an embodiment herein, the endometrial stem cells on the T-plate nano-composite implant nano-composite have a viability of 82%.

According to an embodiment herein, the T-plate nano-composite implant has an osteo-conductive property.

According to an embodiment herein, the nano-composite is non-toxic and environmental friendly.

According to an embodiment herein, a method for synthesizing biodegradable and biocompatible nano-composite T-plate implant is provided. The method comprises synthesizing hydroxyapatite (HAp) nanoparticles, poly lactic glycolic acids (PLGA) and HAp nano composite implant. The casting of the poly lactic glycolic acids (PLGA) and HAp nano composite are done in a mold to obtain a T-plate nano composite. An endometrial stem cell from an epithelial cell lining from uterus is isolated and cultured. The endometrial stem cells are implanted on the nano-composite implant.

According to an embodiment herein, the hydroxyapatite (HAp) nanoparticles are synthesized by a precipitation method or a mechanical-chemical method.

According to an embodiment herein, the T-plate implant from poly lactic glycolic acids (PLGA) and HAp nano composite is synthesized by a melting/casting method or a solution making/casting method.

According to an embodiment herein, the synthesis of hydroxyapatite (HAp) nanoparticles comprises the following steps. A solution of a diammonium hydrogen phosphate [(NH₄)₂.HPO₄] and a calcium nitrate 4-hydrogen [Ca(NO₃)₂.4H₂O] is prepared. The concentration of the diammonium hydrogen phosphate solution is 0.09M, and the concentration of the calcium nitrate 4-hydrogen solution is 0.15M. The pH of the solution is maintained by adding 1M sodium hydroxide (NaOH) at room temperature. The pH of the diammonium hydrogen phosphate solution and calcium nitrate 4-hydrogen solution is within the range of 10-11. The diammonium hydrogen phosphate solution is added into calcium nitrate solution by drop-wise to obtain a hydroxyapatite precipitate and the hydroxyapatite is precipitated. The hydroxyapatite (HAp) preceipitate is kept at a room temperature for 22 hours on a magnetic stirrer rotated with 750 rpm. The hydroxyapatite (HAp) precipitate centrifuged at 300 rpm for 3 minutes. The hydroxyapatite (HAp) precipitate is washed with de-ionized water. The hydroxyapatite (HAp) is freeze-dried at a temperature range of −40° C. to −50° C. to obtain a hydroxyapatite powder. The hydroxyapatite powder is dried for 24 hours. The dried hydroxyapatite powder is subjecting for calcination in an electrical box furnace at 900° C. for 1 hour and the hydroxyapatite powder is cooled at a temperature of 5° C./min in air.

According to an embodiment herein, the synthesis of polylactic glycolic acid (PLGA) and a hydroxyapatite (HAp) composite is done by a solvent casting method. The solvent casting method comprises the following steps. A solution of poly lactic glycolic acid (PLGA) at a concentration of 7%-10% w/v in a solvent is prepared and the solvent is selected from a group consisting of a dioxane and a methyl chloride. The nano (HAp) hydroxyapatite powder is added to the PLGA solution, and the concentration of PLGA/HAp nano composite concentration is 25%, the hydroxyapatite powder and PLGA are added a ratio of 30:70. The PLGA/HAp solution is stirred by using a stirrer until a homogenous mixture is obtained. The mixture of PLGA/nano-HAp is transferred to a polyethylene T-shaped mold. The mixture is freeze-dried at a temperature of −45° C. for 36 hour.

According to an embodiment herein, the synthesis of polylactic glycolic acid (PLGA) and a hydroxyl hydroxyapatite (HAp) nano composite is done by a melting casting method. The melting casting method comprises the following steps. The poly lactic glycolic acid (PLGA) is melted by heating. A hydroxyapatite (HAp) nano composite is added into the molten PLGA. The poly lactic glycolic acid (PLGA) is mixed with hydroxyapatite (HAp) nano composite to obtain a mixture. The mixture is cast into a T-plate shaped mold. The mixture is freeze dried at a temperature of −45° C. for 36 hour.

According to an embodiment herein, the synthesis of hydroxyapatite nanoparticles comprises the following steps. A solution of 0.15M calcium chloride (CaCl₂) and a solution of 0.09 M di sodium hydrogen phosphate are prepared. A pH of the solution is maintained at 10-11 by adding 1M sodium hydroxide (NaOH) at room temperature. Disodium hydrogen phosphate solution is added dropwise into calcium nitrate solution to obtain a precipitate of hydroxyapatite. The ageing of the hydroxyapatite precipitate is done for 22 hours at room temperature on magnetic stirrer rotated at 750 rpm. The hydroxyapatite centrifuged at 300 rpm for 3 minutes and washed with a de-ionized water. The washed hydroxyapatite precipitate is freeze-dried at a temperature range of −40° C. to −50° C. to obtain a hydroxyapatite powder. The hydroxyapatite powder is dried for 24 hours. The dried hydroxyapatite powder is subjected for calcinations in an electrical box furnace at 900° C. for 1 hour. The hydroxyapatite powder is cooled at a temperature of 5° C./minute in atmospheric air condition.

According to an embodiment herein, the synthesis of polylactic glycolic acid (PLGA) and a hydroxyapatite (HAp) composite is done by a solvent casting method. The solvent casting method comprises the following steps. A solution of poly lactic glycolic acid (PLGA) is prepared with a concentration of 7%-10% w/v in a solvent. The solvent is selected from a group consisting of a dioxane and a methyl chloride. Nano (HAp) hydroxyapatite powder is added to the PLGA solution, and the concentration of PLGA/HAp nano composite solution is 25%. The hydroxyapatite powder and PLGA are mixed at a ratio of 30:70. The PLGA/HAp solution is stirred until a homogenous mixture is obtained. The mixture of PLGA/nano-HAp is transferred to a polyethylene T-shaped mold. The mixture is freeze dried at a temperature of −45° C. for 36 hour.

According to an embodiment herein, the T-plate nano-composite implant has a Young's modulus of 157 MPa.

According to an embodiment herein, the isolation and culture of an endometrial stem cell comprises the following steps. An endometrial biopsy tissue sample is obtained from a plurality of female patients of childbearing age. The endometrial tissue is centrifuged in a tube with a 15 ml collagenase type IA and a 2 mg/ml of DMEM. The DMEM comprises of 1% antibiotics 100× penicillin, amphotericin and streptomycin. The contents of the centrifuge tube are incubated for 2 hours at 37° C. The endometrial tissue is filtered through the 45 μm and 70 μm filters for obtaining the cells. The filtered endometrial cells are subjected to centrifuging process at 1000 rpm for 15 minutes. The endometrial stem cells are purified with a ficoll purification protocol. The purified endometrial stem cells are incubated in a flask at a temperature of 37° C., 5% CO₂ atmosphere and moisture of 95%.

According to an embodiment herein, the implantation of endometrial stem cells on nano composite implant comprises the following steps. The nanocomposite implant is sterilized with UV light for 1 hour. 9 ml of DMEM with 10% FBS are added to the nano composite implants. The nanocomposite implant is incubated for 45 minutes. 20 μl of endometrial stem cells is inoculated on the nano-composite implant by a sampler and the concentration of endometrial stem cells is 10⁶ cells/cc. The nanocomposite implant with endometrial stem cells is incubated for 48 hour at temperature of 37° C., humidity of 90% and 5% CO₂ atmosphere.

According to one embodiment herein, the biodegradable materials including biodegradable ceramics and polymers are used extensively for different biomedical applications. The main advantage of biodegradable implant is that there is no need for a secondary surgery for removing the implants after healing a bone damage or tissue repair. Due to the extensive varieties of biodegradable polymers and their similarity with natural tissues in terms of properties, the biodegradable polymers have gained more attention. Various degradable polymers are known and are investigated for medical applications such as poly α-hydroxy acids and poly-anhydrides. The FDA has approved poly lactic acid (PLA), polyglycolic acids (PGA) and their copolymer such as poly glycolic-lactic acid (PLGA). The poly glycolic-lactic acid (PLGA) has property of achieving desirable rate of release, desirable rate of degradability and mechanical properties.

According to one embodiment herein, bioceramic implants are commonly used for improving scaffolds for bone repair. The bioactive ceramics based on calcium phosphates (CaP) are extensively used, specifically the bio-ceramics such as tricalcium phosphate (TCP) [Ca₃(PO₄)₂] and hydroxyapatite (HAp) [Ca₁₀(PO₄)₆(OH)₂] are extensively studied for their clinical applications. These bio-ceramics find their applications in orthopedics and dentistry.

According to one embodiment herein, the bio-ceramics with the general chemical formula of M₁₀(ZO₄) 6×2 are called “apatite” and are classified within the hexagonal crystal structure (space group P63/m). In the formula M is substituted by certain groups of atoms such as Ca, Sr, ba, Cd, Pb. In the formula Z is substituted by certain group of atoms such as P, CO₃, V, As, S, Si, and Ge. In the formula X is substituted by certain group of atoms such as OH, CO₃, O, F, Cl, and Br respectively. The hydroxyapatite (HAp) [Ca₁₀(PO₄)₆(OH)₂] is an important member of this family with a crystallographic structure identical to calcified tissues of vertebrates and has been extensively used as bone substitute. The inorganic matrix component of natural bone is based on hydroxyapatite doped with various quantities of cations like Na, K and Mg and anions such as CO₃, SO₄ and F₂.

The porous hydroxyapatite (HAp) finds its applications in maxillofacial surgery as alveolar ridge augments and as bone defect filler. However the HAp is difficult to handle because of its brittleness and low plasticity. Therefore composite of a polymeric phase beside HAp has been considered as bone substitute with better mechanical properties rather than single HAp.

According to one embodiment herein, the biodegradable composite polymers and bio-ceramics are applied for the treatment of the broken bones or damaged tissues. The new biodegradable polymers such as poly glycolic-lactic acid (PLGA) and calcium phosphate ceramics, provide a better control of the macroscopic and microscopic composite structures for the treatment of tissue damage or bone damage. Also the biodegradable polymers are used to reduce the brittleness of conventional ceramics. The biodegradable polymer/ceramic composite bio-material is a promising tool for bone graft and bone regeneration.

The composite materials generally consist of two phases including a matrix phase and a reinforcement phase. The reinforcing phase is in the form of particle, fiber, and flake. The HAp nanoparticles are used as reinforcing agent in order to enhance the mechanical properties of a prepared composite. The HAp particles also provide an osteo-conductive environment for better bone healing.

According to one embodiment herein, poly glycolic lactic acid (PLGA) and hydroxyapatite (HAp) composite or nano-composite are commonly used for making the biocompatible and biodegradable implants. The degradation rate of PLGA is controlled and adjusted by changing the composition of poly lactic acid (PLA) and polyglycolic acid (PGA) in PLGA. The PLGA acts as a matrix. The HAp acts as the reinforcement in the composite. The HAp is an osteoconductive agent and accelerates the rate of bone healing. Further HAp also acts as pH equilibrating agent in the environment where the PLGA-HAp composite is applied. The PLGA degradation leads to acidic products and lowers the acidity of the tissues surrounding the implant. This is harmful for the surrounding tissues, the HAp prevents the acidity.

According to one embodiment herein, the combination of the stem cells along with biodegradable implants accelerates the healing process of broken bones or damaged tissues. The endometrial stem cells are a new source of stem cells. The endometrial stem cells have high regeneration capability. The endometrial stem cells have the following properties of: angiogenesis, differentiation into three cell layers including endoderm (hepatocyte), mesoderm (osteocyte, adipocyte, cardiomycocytes) and ectodermis (neurons).

According to one embodiment herein, a deposition method is performed for the fabrication of nano-hydroxyapatite. The organic and inorganic solvents are selected from a group of methylene chloride, chloroform, dioxane and isopropanol. The composite of PLGA and HAp is prepared by adding them to the solvents. The nano-HAp and polymer suspension is mixed and precipitated. Further the precipitate is dried and the powder of nano composite is obtained. The HAp and PLGA nano composite powder is used for the T-plate synthesis.

These and other aspects of the embodiments herein will be better appreciated and understood when considered in conjunction with the following description and the accompanying drawings. It should be understood, however, that the following descriptions, while indicating the preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flow chart indicating the process of synthesizing hydroxyapatite nanoparticle in the fabrication of a biodegradable and biocompatible nano composite implant, according to an embodiment herein.

FIG. 2 illustrates a flow chart indicating the process of synthesizing a hydroxyapatite nanoparticle with diammonium hydrogen phosphate and calcium nitrate 4-hydrogen in the fabrication of a biodegradable and biocompatible nano composite implant, according to an embodiment herein.

FIG. 3 illustrating a flow chart indicating the process of synthesizing hydroxyapatite nanoparticle with calcium chloride and disodium hydrogen phosphate in the fabrication of a biodegradable and biocompatible nano composite implant according to an embodiment herein.

FIG. 4 illustrates a photograph of the T-plate implant composite, according to one embodiment herein.

FIG. 5 illustrates a surface electron microscope (SEM) image of the nano-hydroxyapatite/PLGA composite T-plate, according to one embodiment herein.

FIG. 6 illustrates a surface electron microscope (SEM) image of the endometrial stem cells cultured on the T-plate implant composite, according to one embodiment herein.

FIG. 7 illustrates a graph indicating the stress-strain characteristics curve of composite T-plate implant, according to one embodiment herein.

Although the specific features of the embodiments herein are shown in some drawings and not in others. This is done for convenience only as each feature may be combined with any or all of the other features in accordance with the embodiments herein

### DETAILED DESCRIPTION OF THE DRAWINGS

In the following detailed description, as reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a biodegradable implant composite for the treatment of injured/damaged tissue especially fractured bones. The biodegradable implant along with stem cells accelerates the healing of the injured/broken tissue such as bone.

A biodegradable and biocompatible nano-composite T-plate implant comprises a polymeric matrix part, a bioceramic part and an endometrial stem cell. The polymeric matrix part comprises poly lactic glycolic acids (PLGA), and the bioceramic part comprises hydroxyapatite (HAp) nanoparticles. The poly lactic glycolic acid (PLGA) acts as a matrix and the hydroxyapatite (HAp) nanoparticles act as reinforcing agents.

According to an embodiment herein, the hydroxyapatite (HAp) nanoparticles have a spherical shape or a needle like shape within the polymeric matrix part.

According to an embodiment herein, the T-plate nano-composite implant has a Young's modulus of 157 MPa.

According to an embodiment herein, the endometrial stem cells on the T-plate nano-composite implant nano-composite have a viability of 82%.

According to an embodiment herein, the T-plate nano-composite implant has an osteo-conductive property.

According to an embodiment herein, the nano-composite is non-toxic and environmental friendly.

According to an embodiment herein, a method for synthesizing biodegradable and biocompatible nano-composite T-plate implant is provided. The method comprises synthesizing hydroxyapatite (HAp) nanoparticles, poly lactic glycolic acids (PLGA) and HAp nano composite implant. The casting of the poly lactic glycolic acids (PLGA) and HAp nano composite are done in a mold to obtain a T-plate nano composite. An endometrial stem cell from an epithelial cell lining from uterus is isolated and cultured. The endometrial stem cells are implanted on the nano-composite implant.

According to an embodiment herein, the hydroxyapatite (HAp) nanoparticles are synthesized by a precipitation method or a mechanical-chemical method.

According to an embodiment herein, the T-plate implant from poly lactic glycolic acids (PLGA) and HAp nano composite is synthesized by a melting/casting method or a solution making/casting method.

According to an embodiment herein, the synthesis of hydroxyapatite (HAp) nanoparticles comprises the following steps. A solution of a diammonium hydrogen phosphate [(NH₄)₂.HPO₄] and a calcium nitrate 4-hydrogen [Ca(NO₃)₂.4H₂O] is prepared. The concentration of the diammonium hydrogen phosphate solution is 0.09M, and the concentration of the calcium nitrate 4-hydrogen solution is 0.15M. The pH of the solution is maintained by adding 1M sodium hydroxide (NaOH) at room temperature. The pH of the diammonium hydrogen phosphate solution and calcium nitrate 4-hydrogen solution is within the range of 10-11. The diammonium hydrogen phosphate solution is added into calcium nitrate solution by drop-wise to obtain a hydroxyapatite precipitate and the hydroxyapatite is precipitated. The hydroxyapatite (HAp) precipitated is kept at a room temperature for 22 hours on a magnetic stirrer rotated with 750 rpm. The hydroxyapatite (HAp) precipitate centrifuged at 300 rpm for 3 minutes. The hydroxyapatite (HAp) precipitate is washed with de-ionized water. The hydroxyapatite (HAp) is freeze-dried at a temperature range of −40° C. to −50° C. to obtain a hydroxyapatite powder. The hydroxyapatite powder is dried for 24 hours. The dried hydroxyapatite powder is subjecting for calcination in an electrical box furnace at 900° C. for 1 hour and the hydroxyapatite powder is cooled at a temperature of 5° C./min in air.

According to an embodiment herein, the synthesis of polylactic glycolic acid (PLGA) and a hydroxyapatite (HAp) composite is done by a solvent casting method. The solvent casting method comprises the following steps. A solution of poly lactic glycolic acid (PLGA) at a concentration of 7%-10% w/v in a solvent is prepared and the solvent is selected from a group consisting of a dioxane and a methyl chloride. The nano (HAp) hydroxyapatite powder is added to the PLGA solution, and the concentration of PLGA/HAp nano composite concentration is 25%, the hydroxyapatite powder and PLGA are added a ratio of 30:70. The PLGA/HAp solution is stirred by using a stirrer until a homogenous mixture is obtained. The mixture of PLGA/nano-HAp is transferred to a polyethylene T-shaped mold. The mixture is freeze-dried at a temperature of −45° C. for 36 hour.

According to an embodiment herein, the synthesis of polylactic glycolic acid (PLGA) and a hydroxyl hydroxyapatite (HAp) nano composite is done by a melting casting method. The melting casting method comprises the following steps. The poly lactic glycolic acid (PLGA) is melted by heating. A hydroxyapatite (HAp) nano composite is added into the molten PLGA. The poly lactic glycolic acid (PLGA) is mixed with hydroxyapatite (HAp) nano composite to obtain a mixture. The mixture is cast into a T-plate shaped mold. The mixture is freeze dried at a temperature of −45° C. for 36 hour.

According to an embodiment herein, the synthesis of hydroxyapatite nanoparticles comprises the following steps. A solution of 0.15M calcium chloride (CaCl₂) and a solution of 0.09 M di sodium hydrogen phosphate are prepared. A pH of the solution is maintained at 10-11 by adding 1M sodium hydroxide (NaOH) at room temperature. Disodium hydrogen phosphate solution is added dropwise into calcium nitrate solution to obtain a precipitate of hydroxyapatite. The ageing of the hydroxyapatite precipitate is done for 22 hours at room temperature on magnetic stirrer rotated at 750 rpm. The hydroxyapatite centrifuged at 300 rpm for 3 minutes and washed with a de-ionized water. The washed hydroxyapatite precipitate is freeze-dried at a temperature range of −40° C. to −50° C. to obtain a hydroxyapatite powder. The hydroxyapatite powder is dried for 24 hours. The dried hydroxyapatite powder is subjected for calcinations in an electrical box furnace at 900° C. for 1 hour. The hydroxyapatite powder is cooled at a temperature of 5° C./minute in atmospheric air condition.

According to an embodiment herein, the synthesis of polylactic glycolic acid (PLGA) and a hydroxyapatite (HAp) composite is done by a solvent casting method. The solvent casting method comprises the following steps. A solution of poly lactic glycolic acid (PLGA) is prepared with a concentration of 7%-10% w/v in a solvent. The solvent is selected from a group consisting of a dioxane and a methyl chloride. Nano (HAp) hydroxyapatite powder is added to the PLGA solution, and the concentration of PLGA/HAp nano composite solution is 25%. The hydroxyapatite powder and PLGA are mixed at a ratio of 30:70. The PLGA/HAp solution is stirred until a homogenous mixture is obtained. The mixture of PLGA/nano-HAp is transferred to a polyethylene T-shaped mold. The mixture is freeze dried at a temperature of −45° C. for 36 hour.

According to an embodiment herein, the T-plate nano-composite implant has a Young's modulus of 157 MPa.

According to an embodiment herein, the isolation and culture of an endometrial stem cell comprises the following steps. An endometrial biopsy tissue sample is obtained from a plurality of female patients of childbearing age. The endometrial tissue is centrifuged in a tube with a 15 ml collagenase type IA and a 2 mg/ml of DMEM. The DMEM comprises of 1% antibiotics 100× penicillin, amphotericin and streptomycin. The contents of the centrifuge tube are incubated for 2 hours at 37° C. The endometrial tissue is filtered through the 45 μm and 70 μm filters for obtaining the cells. The filtered endometrial cells are subjected to centrifuging process at 1000 rpm for 15 minutes. The endometrial stem cells are purified with a ficoll purification protocol. The purified endometrial stem cells are incubated in a flask at a temperature of 37° C., 5% CO₂ atmosphere and moisture of 95%.

According to an embodiment herein, the implantation of endometrial stem cells on nano composite implant comprises the following steps. The nanocomposite implant is sterilized with UV light for 1 hour. 9 ml of DMEM with 10% FBS are added to the nano composite implants. The nanocomposite implant is incubated for 45 minutes. 20 μl of endometrial stem cells is inoculated on the nano-composite implant by a sampler and the concentration of endometrial stem cells is 10⁶ cells/cc. The nanocomposite implant with endometrial stem cells is incubated for 48 hour at temperature of 37° C., humidity of 90% and 5% CO₂ atmosphere.

According to one embodiment herein, the biodegradable materials including biodegradable ceramics and polymers are used extensively for different biomedical applications. The main advantage of biodegradable implant is that there is no need for a secondary surgery for removing the implants after healing a bone damage or tissue repair. Due to the extensive varieties of biodegradable polymers and their similarity with natural tissues in terms of properties, the biodegradable polymers have gained more attention. Various degradable polymers are known and are investigated for medical applications such as poly α-hydroxy acids and poly-anhydrides. The FDA has approved poly lactic acid (PLA), polyglycolic acids (PGA) and their copolymer such as poly glycolic-lactic acid (PLGA). The poly glycolic-lactic acid (PLGA) has property of achieving desirable rate of release, desirable rate of degradability and mechanical properties.

According to one embodiment herein, bioceramic implants are commonly used for improving scaffolds for bone repair. The bioactive ceramics based on calcium phosphates (CaP) are extensively used, specifically the bio-ceramics such as tricalcium phosphate (TCP) [Ca₃(PO₄)₂] and hydroxyapatite (HAp) [Ca₁₀(PO₄)₆(OH)₂] are extensively studied for their clinical applications. These bio-ceramics find their applications in orthopedics and dentistry.

According to one embodiment herein, the bio-ceramics with the general chemical formula of M₁₀(ZO₄)6X₂ are called “apatite” and are classified within the hexagonal crystal structure (space group P63/m). In the formula M is substituted by certain groups of atoms such as Ca, Sr, ba, Cd, Pb. In the formula Z is substituted by certain group of atoms such as P, CO₃, V, As, S, Si, Ge. In the formula X is substituted by certain group of atoms such as OH, CO₃, O, F, Cl, Br respectively. The hydroxyapatite (HAp) [Ca₁₀(PO₄)₆(OH)₂] is an important member of this family with a crystallographic structure identical to calcified tissues of vertebrates and have been extensively used as bone substitute. The inorganic matrix component of natural bone is based on hydroxyapatite doped with various quantities of cations like Na, K and Mg and anions such as CO₃, SO₄ and F₂.

The porous hydroxyapatite (HAp) finds its applications in maxillofacial surgery as alveolar ridge augments and as bone defect filler. However the HAp is difficult to handle because of its brittleness and low plasticity. Therefore composite of a polymeric phase beside HAp has been considered as bone substitute with better mechanical properties rather than single HAp.

According to one embodiment herein, the biodegradable composite polymers and bio-ceramics are applied for the treatment of the broken bones or damaged tissues. The new biodegradable polymers such as poly glycolic-lactic acid (PLGA) and calcium phosphate ceramics, provide a better control of the macroscopic and microscopic composite structures for the treatment of tissue damage or bone damage. Also the biodegradable polymers are used to reduce the brittleness of conventional ceramics. The biodegradable polymer composites/ceramic bio-material is a promising tool for bone graft and bone regeneration.

The composite materials generally consist of two phases including a matrix phase and a reinforcement phase. The reinforcing phase is in the form of particle, fiber, and flake. The HAp nanoparticles are used as reinforcing agent in order to enhance the mechanical properties of a prepared composite. The HAp particles also provide an osteo-conductive environment for better bone healing.

According to one embodiment herein, poly glycolic lactic acid (PLGA) and hydroxyapatite (HAp) composite or nano-composite are commonly used for making the biocompatible and biodegradable implants. The degradation rate of PLGA is controlled and adjusted by changing the composition of poly lactic acid (PLA) and polyglycolic acid (PGA) in PLGA. The PLGA acts as a matrix. The HAp acts as the reinforcement in the composite. The HAp is an osteoconductive agent and accelerates the rate of bone healing. Further HAp also acts as pH equilibrating agent in the environment where the PLGA-HAp composite is applied. The PLGA degradation leads to acidic products and lowers the acidity of the tissues surrounding the implant. This is harmful for the surrounding tissues, the HAp prevents the acidity.

According to one embodiment herein, the combination of the stem cells along with biodegradable implants accelerates the healing process of broken bones or damaged tissues. The endometrial stem cells are a new source of stem cells. The endometrial stem cells have high regeneration capability. The endometrial stem cells have the following properties of: angiogenesis, differentiation into three cell layers including endoderm (hepatocyte), mesoderm (osteocyte, adipocyte, cardiomycocytes) and ectodermis (neurons).

According to one embodiment herein, a deposition method is performed for the fabrication of nano-hydroxyapatite. The organic and inorganic solvents are selected from a group of methylene chloride, chloroform and dioxane iso propanol. The composite of PLGA and HAp is prepared by adding them to the solvents. The nano-HAp and polymer suspension is mixed and precipitated. Further the precipitate is dried and the powder of nano composite is obtained. The HAp and PLGA nano composite powder is used for the T-plate synthesis.

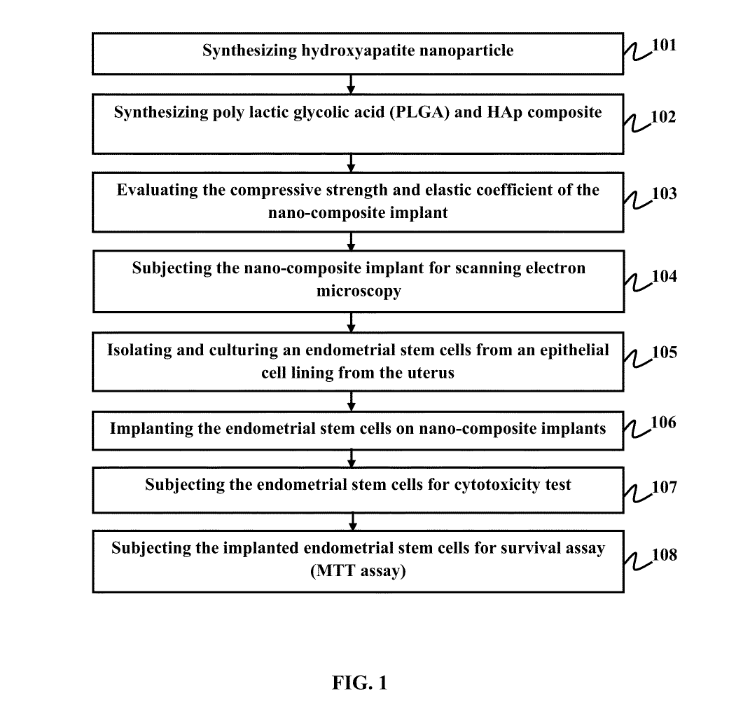

FIG. 1 illustrates a flow chart indicating the process of synthesizing a hydroxyapatite nanoparticle, according to an embodiment herein. The method for the synthesis of biodegradable and biocompatible T-plate nano-composite implant comprises the following steps. The hydroxyapatite (HAp) nanoparticles are synthesized (101). Poly lactic glycolic acids (PLGA) and HAp nano composite are synthesized (102). The compressive strength and elastic coefficient of the nano-composite implant are evaluated (103). The nano-composite implant is subjected to a scanning electron microscopy (104). The isolation and culture of an endometrial stem cell from an epithelial cell lining from the uterus is carried out (105). The endometrial stem cells are implanted on nano-composite implant (106). Also the endometrial stem cells are subjected for cytotoxicity test (107). Further the implanted endometrial stem cells are subjected for a survival assay (MTT assay) (108).

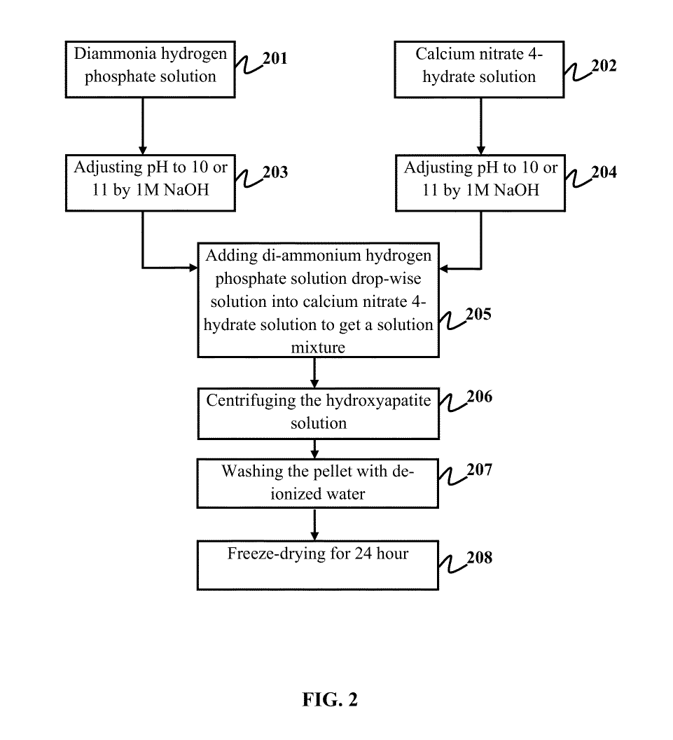

FIG. 2 illustrates a flow chart explaining the process of synthesizing hydroxyapatite nanoparticle with diammonium hydrogen phosphate and calcium nitrate 4-hydrogen, according to an embodiment herein. For the synthesis of hydroxyapatite nanoparticle a solution of 0.09M diammonium hydrogen phosphate solution [(NH₄)₂.HPO₄] is prepared (201) and 0.15M calcium nitrate 4-hydrogen solution [Ca(NO₃)₂.4H₂O] is prepared (202). The pH of the diammonium hydrogen phosphate solution is maintained at 10 or 11 by adding a 1M sodium hydroxide (NaOH) solution at a room temperature (203). The pH of the calcium nitrate 4-hydrogen solution is maintained at 10 or 11 by adding the 1M sodium hydroxide (NaOH) solution at the room temperature (204). The diammonium hydrogen phosphate solution is added dropwise into the calcium nitrate solution to get a solution mixture (205). The hydroxyapatite is precipitated. The precipitate is aged for 22 hours at the room temperature on a magnetic stirrer rotated at a speed of 750 rpm. The hydroxyapatite (HAp) solution is centrifuged at 300 rpm for 3 minutes (206). The hydroxyapatite pellet is washed with de-ionized water (207). The hydroxyapatite is freeze-dried at a temperature range of −40° C. to −50° C. (208). The hydroxyapatite powder is dried for 24 hours and the dried hydroxyapatite powder is subjected for a calcination in an electrical box furnace at 900° C. for 1 hour. The hydroxyapatite powder is cooled at a temperature of 5° C./min air condition. The chemical composition and the grain size of the hydroxyapatite composite nanioparticles are analyzed by a transmission electron microscopy (TEM), an X-ray diffraction (XRD) and a Fourier transform infrared spectroscopy (FTIR).

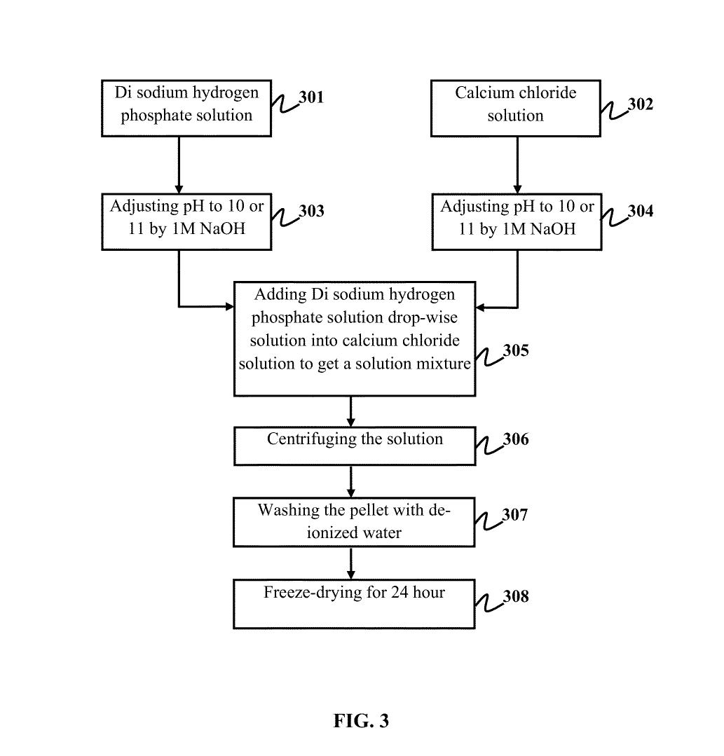

FIG. 3 illustrates a flow chart explaining the process of synthesizing hydroxyapatite nanoparticle with calcium chloride and di sodium hydrogen phosphate, according to an embodiment herein. For the preparation of hydroxyapatite nanoparticle, a solution of 0.15M calcium chloride (CaCl₂) is prepared (301) and a solution of 0.09 M di sodium hydrogen phosphate is prepared (302). The pH of the di sodium hydrogen phosphate solution is maintained at 10-11 by adding a 1M sodium hydroxide (NaOH) solution at a room temperature (303). The pH of the calcium chloride solution is maintained at 10-11 by adding the 1M sodium hydroxide (NaOH) solution at a room temperature (304). The solution of disodium hydrogen phosphate solution is added dropwise into calcium nitrate solution to get a solution mixture (305). The hydroxyapatite is precipitated. The hydroxyapatite precipitate solution is aged for 22 hours at room temperature on magnetic stirrer rotated at a speed of 750 rpm. The hydroxyapatite solution is centrifuged at 300 rpm for 3 minutes (306). The pellet is washed with de-ionized water (307). The hydroxyapatite powder is freeze-dried the at a temperature range of −40° C. to −50° C. for 24 hours (308). The dried hydroxyapatite powder is subjected for calcination in an electrical box furnace at 900° C. for 1 hour. The hydroxyapatite powder is cooled at a temperature of 5° C./minute air condition. The chemical composition and grain size of the hydroxyapatite composite nanoparticle is analyzed by a transmission electron microscope (TEM), a X-ray diffraction (XRD) and a Fourier transform infrared spectroscopy (FTIR).

### EXPERIMENTAL DETAILS

### Example 1

### Hydroxyapatite Nanaoparticles Synthesis

For the synthesis of nanocrystalline hydroxyapatite (HA) powder the solutions of 0.09M diammonium hydrogen phosphate solution [(NH₄).2HPO₄] and 0.15 M calcium nitrate 4-hydrogen solution [Ca(NO₃)₂.4H₂O] were prepared and the pH of both the solutions were brought to 10-11 by adding 1M sodium hydroxide solution (NaOH) at room temperature. The phosphate solution was added drop-wise into calcium nitrate solution, resulting in the precipitation of hydroxyapatite. The ratio of calcium and phosphorus in the initial solution is chosen so that the molar ratio of calcium/phosphorus was 1.67. The precipitate was aged for 22 hours at room temperature on the magnetic stirrer (750 rpm). In the next step, the precipitated hydroxyapatite was centrifuged and then washed with de-ionized water. The solution was centrifuged at 300 rpm for 3 minutes and was transferred to freeze drying machine with a temperature range of approximately −40° C. to −50° C. The process of centrifuging and washing were carried three times. The resulting hydroxyapatite powder was dried in a freeze-drier system for 24 hour. Finally, the dried hydroxyapatite powder was calcined in an electrical box furnace at 900° C. for 1 hour. The hydroxyapatite powder was cooled at a temperature of 5° C./min air condition. In order to analyze the chemical composition and grain size of the synthesized hydroxyapatite nanoparticles transmission electron microscopy (TEM), X-ray diffraction (XRD) and Fourier transform infrared spectroscopy (FTIR) techniques were used.

Construction of Composite Implants Copolymer Poly Lactic Glycolic Acids (PLGA) Nano Hydroxyapatite (HAp) (PLGA/Nano-Hydroxyapatite) [Solvent Casting Method]:

The first solution of poly lactic glycolic acid (PLGA) was prepared at a concentration of 7%-10% w/v in a solvent such as dioxane and methyl chloride. In order to prepare the composite of PLGA/HAp with weight percentage of 25%, nano-hydroxyapatite (nano-HAp) powder was added to the solvent. The hydroxyapatite powder was added to get a ratio of 30:70 (hydroxyapatite/poly lactic glycolic acid) and stirred until a homogenous mixture was obtained. The suspension of nano-HAp and the polymer solution was transferred to a polyethylene mold. The mold with the mixture of nano-HAp and PLGA was subjected to freeze drying at a temperature of −45° C. for 36 hours.

Evaluation of Compressive Strength and Elastic Coefficient of the Nano-Composite Implants:

The compressive strength standard (ASTM F451-86) was provided. The compressive strength was analyzed and measured by Roel-Amstel machine.

Sample Preparation for Scanning Electron Microscope (SEM) Imaging Microscopy:

The samples were fixed and incubated in glutaraldehyde for one hour. Next the sample was incubated for 10-15 minutes in osmium tetroxide 1% prepared in phosphate buffer saline (PBS). Further the sample was incubated for 10-15 minutes in acetone solution. The sample is then dried in freeze drier and sent for scanning electron-imaging (SEM) microscopy. The sample is then photographed with SEM.

Isolation and Culture of Epithelial Cell Lining (Endometrial Stem Cells) from the Uterus:

An endometrial biopsy samples were obtained from female patients of childbearing age. An informed consent was taken from the patients. The patches of endometrial tissues with a scalpel are taken in a centrifuge tube containing 15 ml of collagenase type IA, 2 mg/ml of DMEM (containing antibiotics 1%, 100× penicillin, amphotericin, streptomycin). The centrifuge tube is incubated for 2 hours at 37° C. After digestion of the endometrial tissues, the epithelial cells and stromal cells are filtered through 45 μm and 70 μm filters. After passing the cells through filter the cells are subjected to centrifugation at 1000 rpm for 15 minutes. After centrifugation the cells are purified by ficoll purification protocol. The purified endometrial cells are transferred in a flask and incubated at 37° C., CO₂ 5% and 95% moisture. In order to analyze the endometrial stem cells by flow cytometry the following procedure is followed. The cell suspension (1×10⁷ cell/ml) is taken and incubated for 1 hour with antibodies at 4° C. The antibodies are CD90, anti CD 105, FITC-Phycoerythrin (PE), anti CD 34, anti-human CD 31, or PE-conjugated mouse IgG1, anti CD 61, and anti CD 14. The cell suspension is incubated at a temperature 37° C., CO₂ 5% and 95% moisture for 24 hours. After incubation the cell suspension is washed with phosphate buffer saline and subjected to flow cytometry.

Implantation of Endometrial Cells on Implants-Cytotoxicity Test:

In order to evaluate the cytotoxicity of cells, at first the implants were sterilized with UV light for 1 hour. Further 9 ml of DMEM with FBS 10% was added on the nano-composite implants and was kept in the incubator for 45 minutes. Further 20 μA of endometrial stem cells with a concentration of 10⁶ cell/cc were kept on cast resin and inoculated on the nano-composite implant by a sampler. The nano-composite implant with endometrial stem cells was incubated for 48 hour at 37° C., 90% humidity and 5% CO₂.

Survival of Implanted Endometrial Stem Cells by MTT Assay on Implants:

The appropriate number of cells (preferably, 5000 cells per well) were added to each well and the cells were allowed to grow after attaching with bottom of the plate. Then a test and a control are chosen and a suitable amount of mitogen or drug is added for testing. For achieving the desired affect the plate is incubated. The cells are then subjected to calorimetric analysis. For the calorimetric analysis the formazan crystals are dissolved in dimethyl sulfoxide (DMSO) and the optical density is measured at a wavelength of 570 nm. A standard curve is plotted to calculate the number of viable cells.

### Example 2

### Hydroxyapatite Nanoparticles Synthesis

For the synthesis of nanocrystalline hydroxyapatite (HA) powder the solutions of 0.15M calcium chloride (CaCl₂) and 0.09 M disodium hydrogen phosphate solution [(Na₂HPO₄] were prepared and the pH of both the solutions were brought to 10-11 by adding 1M sodium hydroxide solution (NaOH) at room temperature. The phosphate solution was added drop-wise into calcium nitrate solution, resulting in the precipitation of hydroxyapatite. The ratio of calcium and phosphorus in the initial solution is chosen so that the molar ratio of calcium/phosphorus was 1.67. The precipitate was aged for 22 hours at room temperature on the magnetic stirrer rotated at 750 rpm. In the next step, the precipitated hydroxyapatite was centrifuged and then washed with de-ionized water. The solution was centrifuged at 300 rpm for 3 minutes and was transferred to freeze drying machine with a temperature range of approximately −40° C. to −50° C. The process of centrifuging and washing were carried three times. The resulting hydroxyapatite powder was dried in a freeze-drier system for 24 hour. At last, the dried hydroxyapatite powder was calcined in an electrical box furnace at 900° C. for 1 hour. The hydroxyapatite powder was cooled at a temperature of 5° C./min air condition. In order to analyze the chemical composition and grain size of the synthesized hydroxyapatite nanoparticles transmission electron microscopy (TEM), X-ray diffraction (XRD) and Fourier transform infrared spectroscopy (FTIR) techniques are used.

Construction of Composite Implants Copolymer Poly Lactic Glycolic Acids (PLGA) Nano Hydroxyapatite (PLGA/HAp) [Casting Method for Making Implants]:

In the casting method the polymer PLGA and HAp powder are mixed together in a ratio of 80:20. The mixture is subjected to conventional oven at a temperature of 150° C. After melting and mixing with a tool like castanets, the liquid is poured into aluminum metal frame.

Composite Scaffold Made of Copolymer (PLGA/Nano-HAp):

Scaffolds prepared by solvent method are used for molding. For this purpose dioxane is used as a solvent for dissolving poly lactic glycolic acid (PLGA). The PLGA solution is prepared in dioxane with a concentration of 7% w/v. For the composite of PLGA/nano-HAp preparation, the nano-HAp was added to the solvent. The nano-HAp with soluble polymer solution and suspension was transferred to polyethylene molds. The samples were assigned to the porous structure for 36 hours under freeze drying temperature of −45° C.

Evaluation of Compressive Strength and Elastic Coefficient of the Nanocomposite Implants:

The compressive strength standard (ASTM F451-86) was provided. The compressive strength was analyzed and measured by Roel-Amstel machine.

Sample Preparation for Scanning Electron Microscope (SEM) Imaging Microscopy:

The samples were fixed and incubated in glutaraldehyde for one hour. Next the sample was incubated for 10-15 minutes in osmium tetraoxide 1% solution prepared in phosphate buffer saline (PBS). Further the sample was incubated for 10-15 minutes in acetone solution. The sample is then dried in freeze drier and sent for scanning electron-imaging (SEM) microscopy. The sample is then photographed with a SEM.

Isolation and Culture of Epithelial Cell Lining (Endometrial Stem Cells) from the Uterus:

An endometrial biopsy samples were obtained from female patients of childbearing age. An informed consent was taken from the patients. The patches of endometrial tissues with a scalpel are taken in a centrifuge tube containing 15 ml of trypsin, 2 mg/ml of DMEM (containing antibiotics 1%, 100× penicillin, amphotericin, streptomycin). The centrifuge tube is incubated for 2 hours at 37° C. After digestion of the endometrial tissues, the epithelial cells and stromal cells are filtered through 45 μm and 70 μm filters. After passing the cells through filter the cells are subjected to centrifugation at 1000 rpm for 15 minutes. After centrifugation the cells are purified by ficoll purification protocol. The purified endometrial cells are transferred in a flask and incubated at 37° C., CO₂ 5% and 95% moisture. In order to analyze the endometrial stem cells by flow cytometry, the following procedure is followed. The cell suspension (1×10⁷ cell/ml) is taken and incubated for 1 hour with antibodies at 4° C. The antibodies are CD 90, anti CD 105, FITC-Phycoerythrin (PE), anti CD 34, anti-human CD 31, or PE-conjugated mouse IgG1, anti CD 61, and anti CD 14. The cell suspension is incubated at a temperature 37° C., CO₂ 5% and 95% moisture for 24 hours. After incubation the cell suspension is washed with phosphate buffer saline and subjected to flow cytometry.

Implantation of Endometrial Cells on Implants-Cytotoxicity Test:

In order to evaluate the cytotoxicity of cells, at first the implants were sterilized with UV light for 1 hour. Further, 9 ml DMEM with FBS 10% was added into the nanocomposite implants and was kept in the incubator for 45 minutes. Further 20 μl of endometrial stem cells with a concentration of 10⁶ cell/cc were kept on cast resin and inoculated on the nano-composite implant by a sampler. The nano-composite implant with endometrial stem cells was incubated for 48 hour at 37° C., 90% humidity and 5% CO₂.

Survival of Implanted Endometrial Stem Cells by MTT Assay on Implants:

The appropriate number of cells (preferably, 5000 cells per well) were added to each well and the cells were allowed to grow after attaching with bottom of the plate. Then a test and a control are chosen and a suitable amount of mitogen or drug is added for testing. For achieving the desired effect, the plate is incubated. The cells are then subjected to calorimetric analysis. For the calorimetric analysis the formazan crystals are dissolved in dimethyl sulfoxide (DMSO) and the optical density is measured at a wavelength of 570 nm. A standard curve is plotted to calculate the number of viable cells. The MTT assay illustrated that 82% of the endometrial stem cells are viable. Also the MTT assay and SEM analysis showed that the endometrial stem cells had 82% cell viability on T-plate. The SEM figures also showed strong attachment potential of cells on T-plate.

The synthetic hydroxyapatite is suitable for bone tissue engineering in combination with poly lactic glycolic acid (PLGA). The synthetic hydroxyapatite is bioactive, non-toxic, non immunogenic and osteo-conductive. The nano-structured hydroxyapatite with biocompatible PLGA provides desirable features including osteoblast adhesion, proliferation, differentiation, mineral deposition on the surface of bone leading to the formation of new bone in short time.

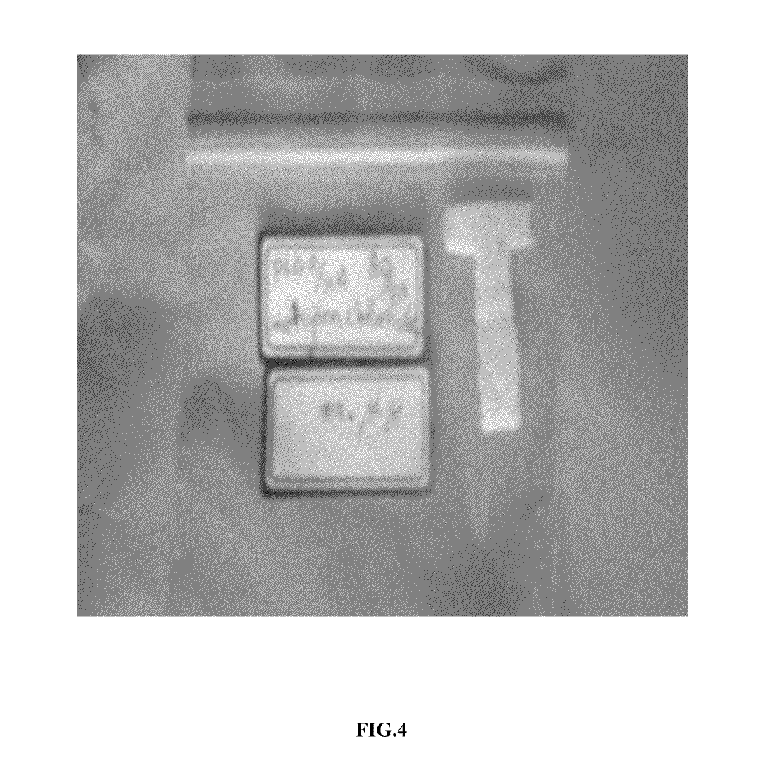

FIG. 4 illustrates a photograph of the T-plate implant composite, according to one embodiment herein. The T-plate is made out of biodegradable copolymer of poly lactic acid (PLA) and poly glycolic acid (PGA). The biodegradable copolymers are selected because of their flexible nature and biocompatibility.

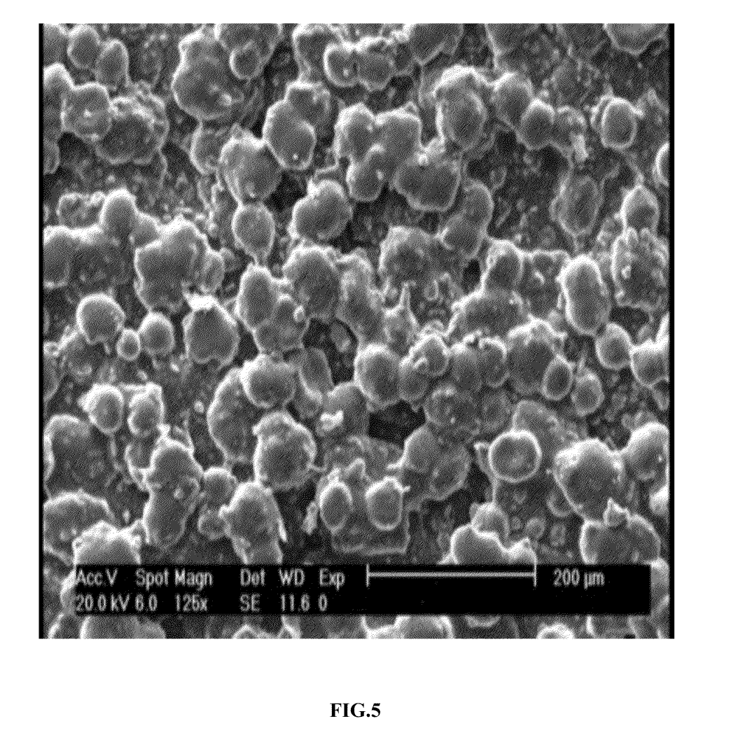

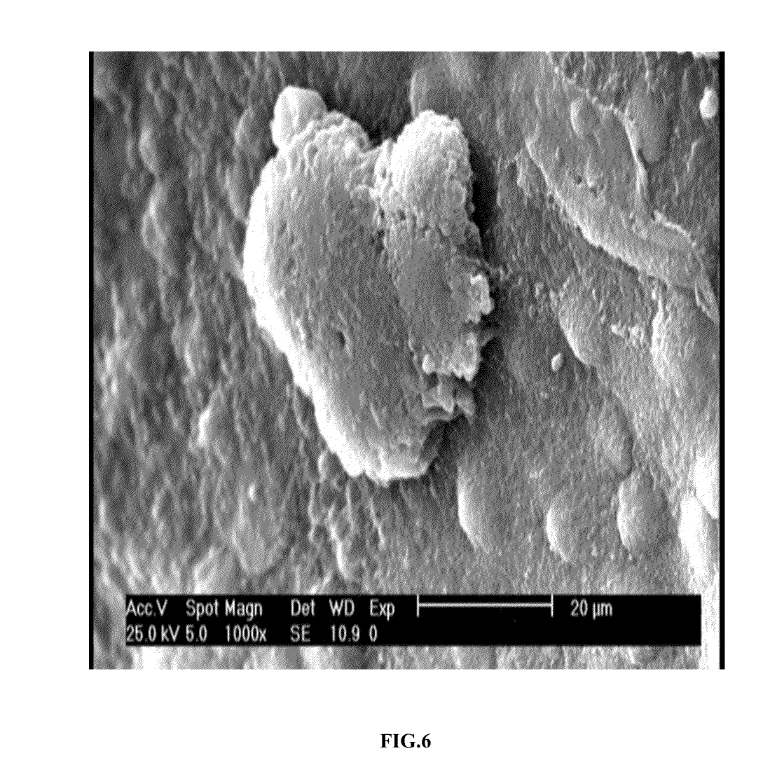

FIG. 5 illustrates a surface electron microscope (SEM) image indicating the nano-hydroxyapatite/PLGA composite T-plate, according to one embodiment herein. For the surface electron microscope (SEM) analysis the T-plate is incubated in glutaraldehyde for one hour. The T-plate is next incubated in 1% osmium tetroxide for 10-15 minutes. The osmium tertoxide is dissolved in phosphate buffer saline. Further the T-plate is incubated in acetone solution for 10-15 minutes followed by freeze drying. The SEM image in FIG. 6 illustrates that the nano-hydroxyapatite/PLGA composite T-plate particle exhibit spherical shape.

FIG. 6 illustrates a surface electron microscope (SEM) image illustrating the endometrial stem cells cultured on the implant composite, according to one embodiment herein. The FIG. 6 shows that the endometrial stem cells are attached with the T-plate implant composite. FIG. 6 also illustrates that the endometrial stem cell also proliferate on the T-plate implant composite. The analysis also illustrates that 82% of endometrial stem cells are viable on T-plate nano-composite implant. The SEM analysis also illustrates strong attachment potential of endometrial stem cells on T-plate.

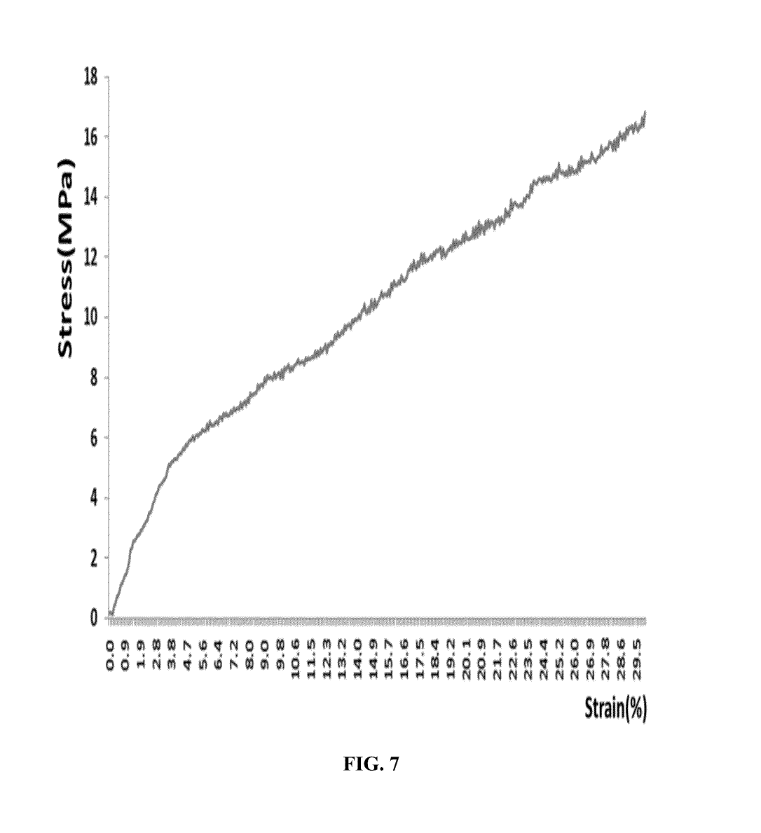

FIG. 7 shows a graph illustrating the stress-strain curve of implant composite, according to one embodiment herein. The stress-strain curve in FIG. 7 illustrates that the T-plate implant composite is suitable for the treatment of bone composite. Further the Young's modulus of the T-plate implant composite is 157 MPa.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments, which as a matter of language might be said to fall there between.

What is claimed is:

1. A biodegradable and biocompatible nano-composite T-plate implant comprises:
a polymeric matrix part;
a bioceramic part; and
an endometrial stem cell;
wherein the polymeric matrix part comprises poly lactic glycolic acids (PLGA), wherein the bioceramic part comprises hydroxyapatite (HAp) nanoparticles.

2. The implant according to claim 1, wherein the poly lactic glycolic acid (PLGA) acts as a matrix and the hydroxyapatite (HAp) nanoparticles act as reinforcing agents.

3. The implant according to claim 1, wherein the hydroxyapatite (HAp) nanoparticles have a spherical shape or a needle like shape within the polymeric matrix part.

4. The implant according to claim 1, wherein the T-plate nano-composite implant has a Young's modulus of 157 MPa.

5. The T-plate nano-composite implant according to claim 1, wherein the endometrial stem cells on the nano-composite have a viability of 82%.

6. The T-plate nano-composite implant according to claim 1, wherein the nano-composite has an osteo-conductive property.

7. The T-plate nano-composite implant according to claim 1, wherein the nano-composite is non-toxic.

8. The T-plate nano-composite implant according to claim 1, wherein the nano-composite is environmental friendly.

9. A method for synthesizing biodegradable and biocompatible nano-composite T-plate implant, the method comprising the steps of:
synthesizing hydroxyapatite (HAp) nanoparticles;
synthesizing poly lactic glycolic acids (PLGA) and HAp nano composite implant;
casting the poly lactic glycolic acids (PLGA) and HAp nano composite into a mold;
isolating and culturing an endometrial stem cell from an epithelial cell lining from uterus; and
implanting the endometrial stem cells on nano-composite implant.

10. The method according to claim 9, wherein the hydroxyapatite (HAp) nanoparticles are synthesized by a precipitation method or a mechanical-chemical method.

11. The method according to claim 9, wherein the T-plate implant from poly lactic glycolic acids (PLGA) and HAp nano composite is synthesized by a melting/casting method or a solution making/casting method.

12. The method according to claim 9, wherein the synthesis of a hydroxyapatite (HAp) nanoparticles comprising the steps of:
preparing a solution of a diammonium hydrogen phosphate [(NH₄)₂.HPO₄] and a calcium nitrate 4-hydrogen [Ca(NO₃)₂.4H₂O], wherein the diammonium hydrogen phosphate solution has a concentration of 0.09M, and wherein the calcium nitrate 4-hydrogen solution has a concentration of 0.15M;
maintaining the pH of the solution by adding 1M sodium hydroxide (NaOH) at room temperature; wherein the pH of the diammonium hydrogen phosphate solution and calcium nitrate 4-hydrogen solution is within a range of 10-11;
adding diammonium hydrogen phosphate solution into calcium nitrate solution by drop-wise to obtain a precipitate of hydroxyapatite;
collecting the precipitating the hydroxyapatite;
keeping the hydroxyapatite (HAp) preceipitate at a room temperature for 22 hours on a magnetic stirrer rotated with 750 rpm;
centrifuging the hydroxyapatite (HAp) precipitate at 300 rpm for 3 minutes;
washing the hydroxyapatite (HAp) precipitate with a de-ionized water;
freeze-drying the hydroxyapatite (HAp) at a temperature range of −40° C. to −50° C. to obtain a hydroxyapatite powder;
drying the hydroxyapatite powder for 24 hours;
subjecting the dried hydroxyapatite powder for calcination in an electrical box furnace at 900° C. for 1 hour; and
cooling the hydroxyapatite powder at a temperature of 5° C./min in air.

13. The method according to claim 9, wherein the synthesis of polylactic glycolic acid (PLGA) and a hydroxyapatite (HAp) composite is done by a solvent casting method, wherein the solvent casting method comprising the steps of:
preparing a solution of poly lactic glycolic acid (PLGA) at a concentration of 7%-10% w/v in a solvent, wherein the solvent is selected from a group consisting of a dioxane and a methyl chloride;
adding the nano (HAp) hydroxyapatite powder to the PLGA solution, wherein the concentration of PLGA/HAp nano composite concentration is 25%, and wherein the hydroxyapatite powder and PLGA are added at a ratio of 30:70;
stirring the PLGA/HAp solution by a stirrer until a homogenous mixture is obtained;
transferring the mixture of PLGA/nano-HAp to a polyethylene T-shaped mold; and
freeze-drying the mixture at a temperature of −45° C. for 36 hour.

14. The method according to claim 9, wherein the synthesis of poly lactic glycolic acid (PLGA) and a hydroxy hydroxyapatite (HAp) nano composite is done by a melting casting method, wherein the melting casting method comprising the steps of:
melting the poly lactic glycolic acid (PLGA) by heating;
adding a hydroxyapatite (HAp) nano composite into the molten PLGA;
mixing the poly lactic glycolic acid (PLGA) and hydroxyapatite (HAp) nano composite mixture;
casting mixture into a T-plate shaped mold; and
freeze drying the mixture at a temperature of −45° C. for 36 hour.

15. The method according to claim 9, wherein the synthesis of a hydroxyapatite nanoparticles comprising the steps of:
preparing a solution of 0.15M calcium chloride (CaCl₂) and a solution of 0.09 M di sodium hydrogen phosphate;
maintaining a pH of the solution at 10-11 by adding 1M sodium hydroxide (NaOH) at room temperature;
adding disodium hydrogen phosphate solution by drop-wise into calcium nitrate solution;
precipitating the hydroxyapatite;
ageing the hydroxyapatite precipitate for 22 hours at room temperature on magnetic stirrer rotated at 750 rpm;
centrifuging the hydroxyapatite precipitate at 300 rpm for 3 minutes and washing with a de-ionized water;
freeze-drying the hydroxyapatite precipitate at a temperature range of −40° C. to −50° C. to obtain a hydroxyapatite powder;
drying the hydroxyapatite powder for 24 hours;
subjecting the dried hydroxyapatite powder for calcinations in an electrical box furnace at 900° C. for 1 hour; and
cooling the hydroxyapatite powder at a temperature of 5° C./minute in air.

16. The method according to claim 9 wherein the synthesis of polylactic glycolic acid (PLGA) and a hydroxyapatite (HAp) composite is done by a solvent casting method and wherein the solvent casting method comprising the steps of:
preparing a solution of poly lactic glycolic acid (PLGA) at a concentration of 7%-10% w/v in a solvent, and wherein the solvent is selected from a group consisting of a dioxane and a methyl chloride;
adding nano (HAp) hydroxyapatite powder to the PLGA solution, and wherein the PLGA/HAp nano composite solution has a concentration of 25%, and wherein the hydroxyapatite powder and PLGA are mixed at a ratio of 30:70;
stirring the PLGA/HAp solution until a homogenous mixture is obtained;
transferring the mixture of PLGA/nano-HAp to a polyethylene T-shaped mold; and
freeze drying the mixture at a temperature of −45° C. for 36 hour.

17. The method according to claim 9, wherein the T-plate nano-composite implant has a Young's modulus of 157 MPa.

18. The method according to claim 9, wherein the isolation and culture of an endometrial stem cell comprising the steps of:
obtaining an endometrial biopsy tissue sample from a plurality of female patients of childbearing age;
centrifuging the endometrial tissue with a 15 ml collagenase type IA, a 2 mg/ml of DMEM, and wherein the DMEM comprises of 1% antibiotics 100× penicillin, amphotericin and streptomycin;
incubating the centrifuge tube for 2 hours at 37° C.;
filtering the endometrial tissue through the 45 μm and 70 μm filters for obtaining endometrial cells;
subjecting the filtered cells to centrifuging at 1000 rpm for 15 minutes;
purifying the endometrial stem cells by ficoll purification protocol; and
incubating the purified endometrial stem cells in a flask at a temperature of 37° C., a 5% CO₂ and a 95% moisture.

19. A method according to claim 9, wherein the implantation of endometrial stem cells on nano composite implant comprising the steps of:
sterilizing the nanocomposite implant with UV light for 1 hour;
adding 9 ml of DMEM with FBS 10% on the nano composite implants;
incubating the nanocomposite implant for 45 minutes;
inoculating 20 μl of endometrial stem cells on nano-composite implant by a sampler and wherein the concentration of endometrial stem cells is 10⁶ cells/cc; and
incubating the nanocomposite implant with endometrial stem cells for 48 hour at temperature of 37° C., a 90% humidity and a 5% CO₂.
